(19) United States
(12) Patent Application Publication
(10) Pub. No.: US 2021/0260404 A1
(43) Pub. Date: Aug. 26, 2021

(54) SYSTEM AND METHOD FOR BRAINWAVE STIMULATION USING ALTERED NATURAL STIMULI

(71) Applicant: KOS CONSULTING EHF, Reykjavik (IS)

(72) Inventors: Atli KOSSON, Reykjavik (IS); Bjarki Freyr SVEINBJARNARSON, Reykjavik (IS); Sveinbjorn HOSKULDSSON, Reykjavik (IS)

(21) Appl. No.: 17/253,298

(22) PCT Filed: Jun. 26, 2019
(86) PCT No.: PCT/IB2019/055417

§ 371 Date: Dec. 17, 2020

Related U.S. Application Data
(60) Provisional application No. 62/689,979, filed on Jun. 26, 2018.

(51) Int. Cl. A61N 5/06; A61N 1/04; A61N 1/36

(52) U.S. Cl. CPC A61N 5/0622; A61N 1/0456; A61N 2005/0648; A61N 1/36036; A61N 1/36025

(57) ABSTRACT

A system and method for brainwave stimulation using altered natural stimuli is provided. The system may comprise a control module and a natural stimulus modulator and is configured to alter at least one natural stimulus signal with frequencies configured to induce brainwave stimulation especially in the range effective for Alzheimer's and other neurological pathologies. The system is configured to prolonged use such that subjects are not prohibited from participation in daily activities and therefore the brainwave stimulation is sufficiently prolonged for enhanced effectiveness. The method for brainwave stimulation may comprise the steps of applying alterations to and delivering at least one natural stimulus signal, measuring brainwave signals and adjusting the alterations accordingly.

### FIELD OF THE DISCLOSURE

The disclosure relates to a system for stimulating brainwaves using altered natural stimuli, particularly in the treatment and prevention of Alzheimer's disease and other neurological pathologies.

### BACKGROUND

The brain is composed of billions of interconnected neurons which connect to and communicate with each other through neural networks using both chemical and electrical signaling. An activated neuron can send signals to other neurons that may cause the other neurons to activate or deactivate. Each neuron forms an electromagnetic field that changes when the neuron is activated. The electromagnetic fields of individual neurons combine to form the electromagnetic field of the brain. The coupling between neurons can give rise to synchronized neural activity which may form relatively large changes in the electromagnetic field of the brain. Patterns in the electromagnetic field of the brain are called brainwaves. Brainwaves can be measured externally, for example by electroencephalograms or magnetoencephalograms. An electroencephalogram (EEG) involves placing electrodes on the surface of a subject's head and measuring the voltage between the electrodes. A magnetoencephalogram (MEG) measures changes in the magnetic field outside a subject's head.

As described above, brainwaves result from the neural activity of the brain. The signal characteristics of the brainwaves can therefore change depending on the neural activity. A person's brainwaves may be affected by their actions, thoughts, and state of mind. For instance, brainwaves as measured through EEG change depending on whether the person is awake or asleep. Brainwaves can also change depending on external stimuli such as touch, smell, sound, light and other input fed into the brain through the sensory nervous system.

The characteristics of the brainwaves can be intentionally affected using different methods, such as through brainwave stimulation using mixtures of artificial visual, auditory, and/or other sensory stimuli that are delivered to a person such as a subject receiving neurological treatment in a way that triggers a certain desired response. In particular, applying stimulation at a particular frequency may result in brainwave stimulation at the same frequency.

This type of brainwave stimulation has been used for different purposes, such as for meditation and as an alternative treatment to drug therapy for certain neurological pathologies, such as Alzheimer's disease, depression, ADHD and Parkinson's, to name a few. Experiments using audial stimulation designed to evoke a 40 Hz brainwave response was used on Alzheimer's patients, for example, demonstrated increased performance in cognitive testing. In a study on mice that had been modified to develop Alzheimer's, the study results indicate that strong brainwave stimulation through audial and visual stimulation on the gamma frequency (around 40 Hz) has a direct effect on the concentration of the peptide amyloid-β. This peptide may form plaques in the brain if its concentration is too high. Plaques of amyloid-β are suspected of being largely responsible for the memory loss and cognitive and motor skill loss that are characteristic of Alzheimer's disease.

Even though brainwave stimulation can be advantageous for various applications and has the potential to be used more widely as an alternative or supportive treatment with drugs for different conditions, it can have some downsides. The effect of brainwave stimulation may be weak and may thus require the brainwave stimulation to be applied continuously or for long periods of time in order to have significant benefits for the subject. Existing treatments for conditions that make use of brainwave stimulation by feeding artificial signals to the sensory input of the subject thus limit the subject from using their sensory inputs during the treatment for significant periods of time, in lieu of conducting other normal, daily activities.

Existing treatments accordingly may not be portable or safe to use while on the move. For instance, a system for delivering visual stimulation may require a subject to look at a sequence of flashing lights on a screen or a projecting device for prolonged periods, rather than being able to go about daily activities that require the subject's sight. Thus a subject receiving visual brainwave stimulation cannot use their sight normally during the brainwave-stimulation process, a subject receiving auditory brainwave stimulation cannot hear normally during the brainwave-stimulation process, and a subject receiving tactile brainwave stimulation cannot feel normally during the brainwave-stimulation process.

Another problem that arises with existing methods of brainwave stimulation is the brain's propensity for adaptation: the longer the subject receives brainwave stimulation treatment, the less effective it becomes, because the brain adapts or becomes desensitized to the brainwave stimulation and no longer reacts strongly thereto. This is particularly observed in of existing attempts to provide brainwave stimulation using artificial sensory inputs, as the brain adapts and becomes desensitized quickly (i.e. within 20 minutes) to artificial stimuli.

In view of the foregoing, there is a need for a system and method for providing brainwave stimulation that does not prohibit a subject from engaging in daily activities and does not diminish in efficacy over time through desensitization, but rather that can be used in conjunction and in tandem with natural stimuli, thus not inhibiting a subject's daily activities and not losing its efficacy.

### SUMMARY

The system and method for brainwave stimulation using altered natural stimuli according to embodiments of the disclosure overcomes the problems of brainwave stimulation prohibiting a subject from engaging in normal daily activities by intercepting, augmenting, and/or altering natural stimuli with frequencies and modulations for brainwave stimulation to enhance at least one targeted brainwave pattern such that the subject is not wholly deprived of natural stimuli and such that the subject's brain does not become desensitized to the brainwave stimulation.

In visual brainwave stimulation, the system and method for brainwave stimulation using altered natural stimuli provides, in an embodiment, a stimulating unit comprising a headset with a camera and a display that intercepts, measures, and/or records the environment that the subject is or would be looking at, adds a brainwave-stimulating signal to the measured environment, and then plays the combined picture on the display for the subject. In another embodiment, the headset with camera and display may modulate the measured environment with patterns that produce desired brainwave stimulation. The subject is thereby enabled to go about normal activities with minimal lifestyle disruption and while receiving beneficial brainwave stimulation. The system and method for brainwave stimulation according to embodiments of the disclosure further avoids the problem of the subject's brain adapting to prolonged stimulation by modulating natural stimuli rather than artificial stimuli. Because the brain is provided with interesting and stimulating natural stimuli which are then modulated with desired brainwave-stimulating frequencies, the brain does not adapt to the stimulation and become desensitized thereto as it would with artificial stimuli.

In aural brainwave stimulation, the system and method for brainwave stimulation using altered natural stimuli provides, in an embodiment, a stimulating unit configured for intercepting, measuring, and/or recording the sound environment around the subject, adding the brainwave-stimulation signal to the measured environment, and playing the combined audio for the subject. In another embodiment, the system modulates the measured environment with patterns that cause the desired brainwave stimulation. As with visual-stimulation embodiments, the subject is enabled to go about normal activities with minimal lifestyle disruption and while receiving beneficial brainwave stimulation.

In another embodiment of the system and method for brainwave stimulation using altered natural stimuli, brainwave stimulation is provided to a subject visually by blocking the subject's vision at specified patterns, thus providing brainwave stimulating patterns without depriving the subject of their natural perceptions. In an analogous embodiment, brainwave stimulation is provided to a subject aurally by providing natural sounds to the subject's ears in the form of amplitude modulated sound, with brainwave stimulating patterns shaping the natural soundscape of a subject going about their daily activities.

In other embodiments of the system and method for brainwave stimulation using altered natural stimuli, bone-hearing speakers may deliver altered environmental sounds to stimulate a subject's brainwaves without interfering with everyday activities and without blocking the subject's ears.

In tactile or touch-sensation brainwave stimulation treatments, the system and method for brainwave stimulation using altered natural stimuli provides for the interception and alteration of a subject's natural sensation of touch by intercepting touch pressure (e.g. at the bottom of one or both of a subject's feet), modulating the intercepted pressure with desired frequencies, and feeding the combined signal to the touch-sensitive sensory input of the subject. In an embodiment, a plate or membrane is located between the subject's feet and the ground, which modulates the pressure experienced between the subject's foot and the ground with brainwave-stimulating frequencies. This advantageously allows the subject to go about normal activities while receiving brainwave stimulation and without desensitization to the brainwave stimulation.

In other embodiments of the system and method for brainwave stimulation using altered natural stimuli, touch-sensation brainwave stimulation is delivered through a dynamic fluid-filled membrane between the subject's feet and the ground, the membrane providing desired frequencies of pulses or vibrations to the subject's foot. In other embodiments, touch-sensation brainwave stimulation is provided through a glove which intercepts the pressure encountered by the subject's hands and fingers and modulates the pressure with a desired frequency.

The system may be linked with a wearable sensor, such as an EEG, allowing the brainwave stimulation to be adapted to bring the measured EEG of the subject closer to a desired setpoint. This arrangement may allow, in certain embodiments, for maximization of a relationship such as alignment or coherence between the brainwave stimulation and the EEG or for maximizing or minimizing specific patterns in the EEG. A control module connected to the sensor and the stimulating unit may employ regular or nonlinear filters, adaptive filters, or machine learning methods to optimize the stimulation towards a desired setpoint. The stimulation may be time-varied and may be tailored to a particular subject's dynamic needs and activities. The control module may further create a control signal for controlling the operation of the natural stimulus modulator(s).

The system may be made of one or more stimulating units that are either self-sustaining for measure, processing and control of the stimulation, or the simulating units may form a network using wired or wireless links for communicating data between the stimulating units or between the stimulating units and a central control unit. The stimuli required of the overall system to create a desired pattern of brainwave stimulation may therefore be determined centrally and communicated to the stimulating units forming the system.

The system may communicate over a network to a local server or cloud service for transferring of any commands, settings, or data for any purposes such as data storage, data processing, stimulation intensity and time recording, stimulation settings, and stimulation control for example.

These and other features, aspects, and advantages of the disclosure will become better understood with regard to the following description, appended claims, and accompanying drawings.

### BRIEF DESCRIPTION OF THE DRAWINGS

FIG. 1 is a perspective view of a system for brainwave stimulation using altered natural visual stimuli according to the disclosure.

FIG. 2 is a perspective view of a system for brainwave stimulation using altered natural aural stimuli according to another embodiment of the disclosure.

FIG. 3 is a perspective view of another system for brainwave stimulation using altered natural visual stimuli according to another embodiment of the disclosure.

FIG. 4 is a top view of a system for brainwave stimulation using altered natural tactile stimulation according to another embodiment of the disclosure.

FIG. 5 is a perspective view of a system for brainwave stimulation using altered visual stimulation according to the embodiment of FIG. 1 and FIG. 2 and brainwave feedback according to another embodiment of the disclosure.

FIG. 6 is a diagram of a system for brainwave stimulation according to another embodiment of the disclosure comprising a person wearing simultaneously a stimulation unit for aural and visual stimulation along with a sensor for measuring an EEG signal on the forehead.

FIG. 7 is a flowchart of a method for brainwave stimulating using altered natural stimuli according to the disclosure.

FIG. 8 depicts graphs showing the alignment between applied brainwave stimulation and measured EEG signals in aural stimulation using a system for brainwave stimulation according to embodiments of the disclosure.

FIG. 9 depicts graphs showing the alignment between applied brainwave stimulation and measured EEG signals in visual stimulation using a system for brainwave stimulation according to embodiments of the disclosure.

FIG. 10A depicts a graph showing the alignment between applied brainwave stimulation and measured EEG signals in the first 30 minutes of aural brainwave stimulation using modulated music according to an embodiment.

FIG. 10B depicts a graph showing the alignment between applied brainwave stimulation and measured EEG signals in the last 30 minutes of aural brainwave stimulation using modulated music according to an embodiment.

FIG. 10C depicts a graph showing the alignment between applied brainwave stimulation and measured EEG signals in the entire 85 minutes of aural brainwave stimulation using modulated music according to an embodiment.

FIG. 11A depicts a graph showing the alignment between applied brainwave stimulation and measured EEG signals in the first 30 minutes of aural brainwave stimulation using modulated white noise according to an embodiment.

FIG. 11B depicts a graph showing the alignment between applied brainwave stimulation and measured EEG signals in the last 30 minutes of aural brainwave stimulation using modulated white noise according to an embodiment.

FIG. 11C depicts a graph showing the alignment between applied brainwave stimulation and measured EEG signals in the entire 85 minutes of aural brainwave stimulation using modulated white noise according to an embodiment.

FIG. 12A depicts a graph showing the alignment between applied brainwave stimulation and measured EEG signals in the first 30 minutes of visual brainwave stimulation using modulated dashcam-driving video according to an embodiment.

FIG. 12B depicts a graph showing the alignment between applied brainwave stimulation and measured EEG signals in the last 30 minutes of visual brainwave stimulation using modulated dashcam-driving video according to an embodiment.

FIG. 12C depicts a graph showing the alignment between applied brainwave stimulation and measured EEG signals in the entire 85 minutes of visual brainwave stimulation using modulated dashcam-driving video according to an embodiment.

The drawing figures are not drawn to scale, but instead are drawn to provide a better understanding of the components, and are not intended to be limiting in scope, but to provide exemplary illustrations. The figures illustrate exemplary configurations of brainwave stimulation systems using altered natural stimuli, and in no way limit the structures or configurations of a system and method for brainwave stimulation using altered natural stimuli according to the present disclosure.

### DETAILED DESCRIPTION OF VARIOUS EMBODIMENTS

A better understanding of different embodiments of the disclosure may be had from the following description read in conjunction with the accompanying drawings in which like reference characters refer to like elements.

While the disclosure is susceptible to various modifications and alternative constructions, certain illustrative embodiments are shown in the drawings and are described below in detail. It should be understood, however, that there is no intention to limit the disclosure to the specific embodiments disclosed, but on the contrary, the intention is to cover all modifications, alternative constructions, combinations, and equivalents falling within the spirit and scope of the disclosure.

It will be understood that, unless a term is defined in this disclosure to possess a described meaning, there is no intent to limit the meaning of such term, either expressly or indirectly, beyond its plain or ordinary meaning.

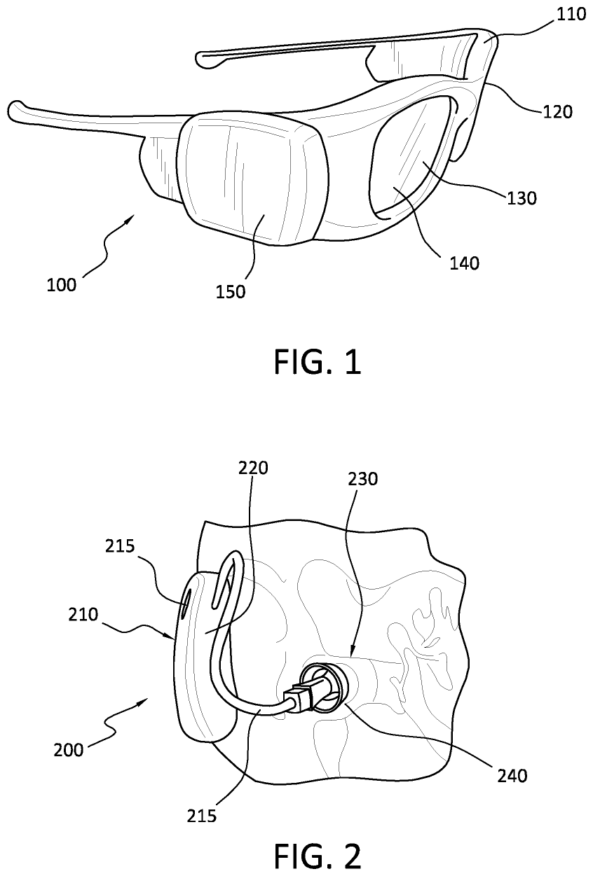

An embodiment of the system and method for brainwave stimulation using altered natural visual stimuli is depicted in FIG. 1. The system 100 stimulates brainwaves using active shutter techniques, allowing the subject to wear the system 100 during normal daily activities and receive effective brainwave stimulation with minimized disruption to the subject's quality of life, thereby overcoming the problems in the art. The system 100 comprises active-shutter eyewear for delivering modulated natural visual stimuli to a subject, including an active-shutter lens frame 110, right and left active-shutter lenses 120, 130, a UV filter 140, and a control module 150 governing the brainwave stimulation activities of the system 100. The control module 150 may comprise a power source that powers the system 100.

The system 100 is configured to block the passage of natural visual stimuli (i.e., light from the natural environment that the subject would see through their eyes) to the subject's eyes at a predetermined or adaptable frequency such that brainwaves are stimulated for treatment of neurological pathologies or other benefits of brainwave stimulation while still providing the natural visual stimuli to the subject such that daily activities are not hampered and such that subject's brain does not adapt to the stimulation, which would otherwise decrease the effectiveness thereof.

In certain embodiments, the natural visual stimuli may be blocked by the right and left active shutter lenses 120, 130 at a rate of, e.g., 40 times per second or 40 Hz, and the system 100 may only allow the natural visual stimuli to enter the subject's eyes for a certain portion of time, e.g. an open/close ratio of 50%. By providing modulations of the natural stimuli at a particular frequency, the brainwaves of the subject may be influenced to have a corresponding frequency, in particular a frequency for stimulating brainwaves. The system 100 may be adaptable to the brightness of natural visual stimuli: for example, the percentage of time that the natural visual stimuli are blocked may be increased when the subject is outdoors and the natural visual stimuli are brighter, and the percentage of time that the natural visual stimuli are blocked may be decreased when the subject is indoors and the natural visual stimuli are less bright.

Because periodic blocking of natural visual stimuli may cause the subject's pupils to dilate larger than their normal state, a UV filter 140 may be provided to protect the eyes from damage resulting from increased exposure of the inner structures of the eye to UV radiation. It will be understood that the system 100 may block the right and left active shutter lenses 120, 130 at a rate greater or less than 40 times per second, such as in a frequency range of between 20 and 80 times per second, and particularly at 30 or 50 times per second in certain embodiments. The system 100 also may allow a lower or higher percentage than 50% of the natural visual stimuli to enter the subject's eyes, as deemed advantageous for a particular subject, a particular environment, or otherwise.

The control module 150 may be arranged as any suitable control element, such as a microprocessor and associated software. The control module 150 may be arranged to control the operation of the active shutter lenses 120, 130 by creating a control signal. The control signal may be delivered to the active shutter lenses 120, 130 and cause the active shutter lenses 120, 130 block light at a desired frequency and to block a desired percentage of the total light. The control signal may be created to effect a targeted brainwave pattern.

It will be understood that the described structures, frequency, open/close ratio, and adaptation to brightness of natural visual stimuli are merely exemplary. Other frequencies, ratios, adaptations, and structures may be provided as suitable and within the spirit and scope of the disclosure. The active-shutter lenses 120, 130 are exemplary and are not limiting. Altered natural visual stimuli may be intercepted, measured, modulated, and/or delivered by any suitable device.

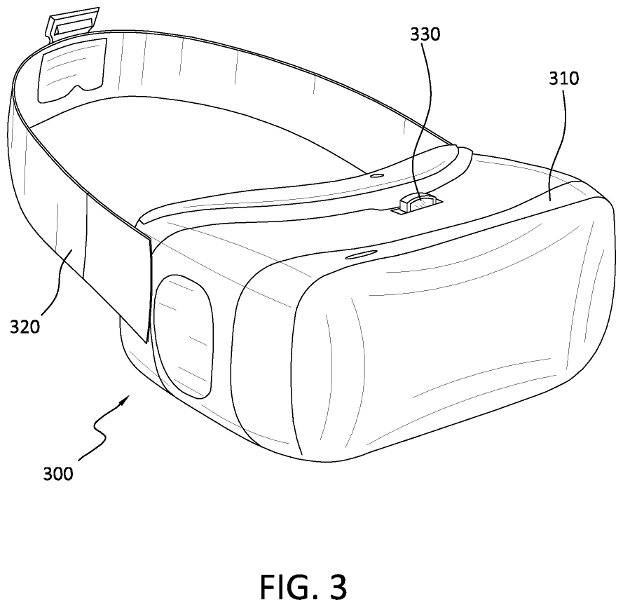

In the embodiment of the system and method for brainwave stimulation using altered natural stimuli illustrated in FIG. 2, natural aural stimuli are altered and utilized to provide brainwave stimulation without disrupting or prohibiting participation in daily activities requiring the subject's hearing and ears. An aural stimulation system 200 is configured to receive natural aural stimuli, i.e. sounds, that a subject would ordinarily hear, modulate the natural aural stimuli with frequencies that stimulate brainwaves, and then feed the altered natural aural stimuli to the subject's ear.

In the illustrated embodiment, system 200 comprises an external component 210 and an internal component 230. The external component 210 is configured to be worn or placed proximate the subject's ear. The external component 210 comprises a microphone 215 and an amplifier 220. The microphone 215 is configured to intercept, measure, and/or record natural aural stimuli and to feed the measured data to the amplifier 220.

The amplifier 220 may comprise a control module configured to modify and amplify the natural aural stimuli measured and/or recorded by the microphone 215. In embodiments, the modulated and amplified sound may be delivered to the subject as amplitude-modulated sound, where the volume of the natural aural stimuli is altered or changed at a desired frequency. In certain embodiments, an aural stimulation system 200 is provided at both ears, with the aural stimuli being mutually modulated at each ear. For example, the system 200 may modulate the natural aural stimuli at a frequency of 40 Hz simultaneously in both ears, or the system 200 may alternate the modulation of the natural aural stimuli between the systems at each of the ears. The description of the amplifier 220 as providing amplitude-modulated sound is merely exemplary and may deliver intercepted natural aural stimuli modulated in any suitable way.

In embodiments, the stimulation may be provided by the system 200 as a difference between the frequencies provided at the left and right ears, e.g. a bi-aural or binaural beats configuration. For example, the system 200 at the left ear provides a frequency of 500 Hz whereas the right ear provides a frequency of 540 Hz, with the resulting difference of 40 Hz evoking and stimulating brainwaves in the desired range, such as at a frequency of 40 Hz.

The internal component 230 comprises a delivery system such as a receiver 240 connected to the external component 210 by a connection component 250. The receiver 240 converts a digital signal received from the modulator/amplifier 220 into an analog sound that is delivered to the subject's ear. Not shown is a power source provided in the external component 210.

In other embodiments of the system 200, the modulator/amplifier 220 adds synthesized sounds to the natural aural stimuli measured by the microphone 215 and amplitude-modulates the sound, so that the combined synthesized sounds and altered natural aural stimuli are presented to the subject through the internal component 230 for increased brainwave stimulation. By providing an aural stimulation system such as the system 200 depicted in FIG. 2, the subject is able to receive sufficiently-long periods of stimulation to receive the desired brainwave-stimulation effect while participating in normal daily activities, as the brainwave stimulation is added to natural aural stimuli without disrupting the subject's use of their ears.

While the system 200 is shown as comprising both an internal and an external component, the depicted embodiment is merely exemplary and may comprise only an external component or only an internal component as suitable.

In yet further embodiments of a system and method for brainwave stimulation, the system may deliver brainwave stimulation through the medium of bone-hearing speakers. In certain embodiments, the system 200 described above may cooperate with bone-hearing speakers to provide further modulation of natural aural stimuli measured and/or recorded by the microphone 215 and/or to provide synthesized sounds to the subject. The use of bone-hearing speakers is particularly advantageous as it allows for the provision of modulated frequencies and synthesized sounds, particularly in combination with natural aural stimuli, without blocking the subject's ears and without otherwise interfering with or disrupting the subject's daily activities. In certain embodiments, bone-hearing speakers may be used in lieu of the receiver 240, thus leaving the subject's ears entirely unchanged and uninhibited.

Another embodiment of the system and method for brainwave stimulation using altered natural stimuli depicted in FIG. 3 augments natural visual stimuli to stimulate brainwaves while allowing the subject to participate in daily activities. The system 300 comprises a headset including a delivery system such as a screen 310 on which altered natural visual stimuli are depicted and delivered to the subject. A camera 330 captures natural visual stimuli, such as the sights that the subject would ordinarily see with their unaided eyes, and the captured natural visual stimuli are then altered, augmented, and/or modulated with frequencies suitable for causing beneficial brainwave stimulation. The system 300 may be secured to the subject via a securing strap 320. Not shown is a power source and a control module regulating the alterations of the captured natural visual stimuli as described in previous embodiments.

In certain embodiments, the system 300 modulates the measured natural visual stimuli at frequencies that induce brainwave stimulation without substantial deprivation of the subject's perception of the natural stimuli. In other embodiments, the system 300 augments the captured natural visual stimuli with prerecorded images and colors that induce brainwave stimulation without substantially altering the subject's perception such that daily activities are prohibited or significantly disrupted. This allows the subject to utilize system 300 for sufficiently long periods of time for effective treatment of neurological pathologies or receiving other benefits of brainwave stimulation.

In further embodiments of a system for brainwave stimulation using altered natural visual stimuli, a set of augmented-reality glasses is provided, allowing the subject to watch and perceive the natural visual stimuli of the subject's physical environment through the glasses. The glasses are configured to display augmented shapes within the visual range of the glasses such that brainwaves are stimulated without requiring the subject to remove themselves from their daily activities.

In another embodiment of a system for brainwave stimulation using altered natural visual stimuli, the above-mentioned augmented reality glasses or a suitable transparent medium may be worn by the subject and a brainwave-stimulating modulation of the transparency of the glasses is effected. An example of a suitable transparent medium is liquid crystal devices. In particular embodiments, the transparent medium is modified by a control module which may, e.g., temporarily dim the medium, either partially or fully, with brainwave-stimulating patterns, such as frequencies in the range of 20 Hz to 80 Hz and/or frequencies evoking brainwaves in the range of 20 Hz to 80 Hz. This arrangement advantageously allows for brainwave stimulation without completely or even substantially depriving the subject of their eyesight and thus their ability to carry on with daily activities while the stimulation is provided.

It will be understood that any of the above-mentioned natural visual stimulation methods may be used in combination for achieving desired brainwave-stimulation effects. For example, a system may incorporate both active shutter lenses, as in the embodiment of FIG. 1, as well as transparency-modulating lenses, to achieve the desired effect. It will be further understood that contact lenses may be used in place of augmented reality glasses, active-shutter technology, or other visual display embodiments.

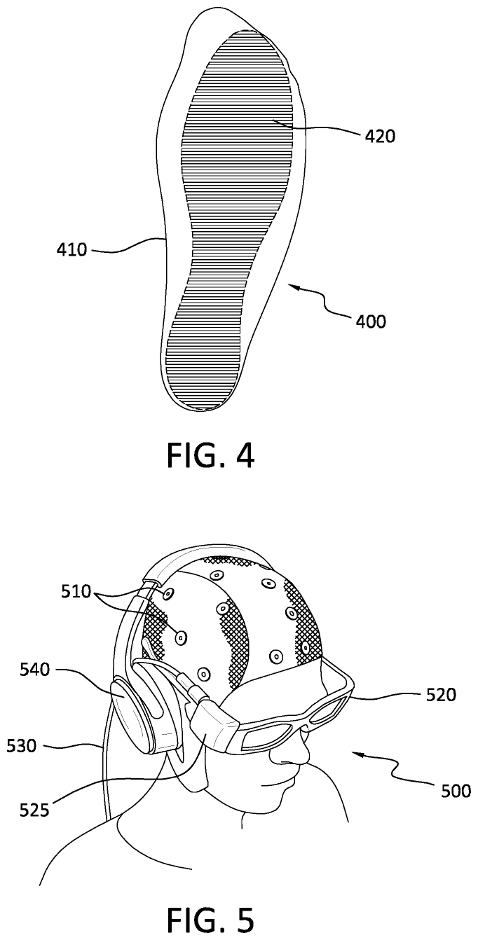

In the embodiment of the system and method for brainwave stimulation using altered natural stimuli depicted in FIG. 4, natural tactile or sensory stimuli are altered to provide brainwave stimulation. The system 400 intercepts touch pressure and modulates the touch pressure with desired frequencies to attain desired brainwave stimulation patterns. The system 400 comprises a stimulation pad 420 that is inserted into a shoe 410 and is configured to contact the bottom of the subject's foot, transmitting pressure and other tactile sensations from the ground or floor to the subject's foot. The stimulation pad 420 may comprise, for example, a plate or a membrane. The membrane may comprise a fluid-filled sole configured to inflate when the foot is lifted and to pulse and/or vibrate in a desired modulation frequency when pressure is placed by the foot on the membrane. As in previous embodiments, the stimulation pad 420 may modulate the pressure felt by a foot at frequencies in a range of 20 Hz to 80 Hz, stimulating and evoking brainwaves at a corresponding frequency. Not shown is a control module and a power source connected to the stimulation pad 420.

In other embodiments of a system for brainwave stimulation using altered natural tactile or sensory stimulation, a glove may be configured to be worn on the hand of the subject, the glove arranged to intercept touch pressure and to transmit the touch pressure to the subject's hand with desired modulation frequencies for brainwave stimulation beneficial for Alzheimer's and other neurological pathologies. The described embodiments are merely exemplary and yet further embodiments of a natural tactile or sensory stimuli-based stimulation system are envisioned. For instance, a garment comprising a stimulation pad arranged proximate a subject's back may be provided and may have a control module configured to modulate touch pressure to the subject's back when the subject is sitting in a chair. Any suitable arrangement of tactile or sensory stimulation components may be utilized in a system according to the disclosure.

It will be understood that any of the above-mentioned embodiments (comprising techniques for altering natural visual, aural, and sensory or tactile stimulation) may be used alone or in any suitable combination to achieve the desired effects. For example, a system according to the disclosure may advantageously incorporate both active-shutter-based visual stimulation according to the embodiment of FIG. 1 with aural stimulation according to the embodiment of FIG. 2 and with tactile stimulation according to the embodiment of FIG. 4 for optimal effect.

While embodiments of the disclosure describe that frequencies of, e.g., 20 Hz-80 Hz may be utilized, it will be understood that the depicted embodiments are not limiting and that any frequency or combination of frequencies may be utilized. For example, a combination of 40 Hz and 1 kHz stimuli may be simultaneously delivered to a subject. In other embodiments, a system for brainwave stimulation using altered natural stimuli according to the disclosure may make use of any combination of suitable types of brainwave-stimulation techniques at any suitable frequency or other metric.

Any of the above-mentioned embodiments may be further used in conjunction with a sensor such as MEG sensors or EEG electrodes to provide feedback-controlled adaptation of the brainwave stimulation based on the subject's response. In the embodiment depicted in FIG. 5, a system for brainwave stimulation using altered natural visual stimulation according to the embodiment depicted in FIG. 1 is combined with EEG feedback. The system 500 comprises an EEG sensor 510 with electrodes arranged to obtain EEG data from a plurality of locations on the subject's head, in particular along the scalp and forehead, and to feed the gathered EEG data to a control module 530. The control module 530 receives the gathered EEG data and adapts the activity of a stimulation apparatus 520 accordingly. It is to be understood that the number of EEG sensors may be more or fewer than the electrodes shown on the picture and may only be located on a portion of the head, such as the forehead. The electrodes may be placed at other suitable locations, such as in a subject's ear.

In certain instances, for example, the control module 530 may compute that the subject's brain has adapted to a particular stimulation pattern (based on a lack of response to stimulation) and then direct the stimulation apparatus 520, which is depicted as a headset for altering natural visual stimuli according to the embodiment of FIG. 1, to change the intensity and/or frequency of stimulating patterns. In other instances, the control module 530 may measure a relationship such as the alignment or coherence between the brainwave stimulation and the measured EEG signal so as to ensure the desired degree of stimulation. The system 500 may also be advantageously used to measure certain patterns in the EEG during or in between stimulation sessions. The feedback loop of system 500 may further be advantageously used to guide the subject's brainwaves to attain a predetermined setpoint value. In yet further embodiments, the feedback loop of system 500 may be arranged to target certain setpoints and/or frequencies at different times or during different activities according to a particular subject's needs.

The feedback loop utilized in embodiments such as system 500 may be based on regular linear or nonlinear filters, adaptive filters, or artificially learned methods. For example, a reinforcement learning algorithm or other machine learning models or algorithms may be utilized in the control module 530 to effectively match the brainwave stimulation from the stimulation apparatus 520 to the subject's response as measured by the EEG sensor 510.

It will be understood that the system 500 depicted in FIG. 5 is not limited to natural visual stimuli, but may also pertain to natural aural and tactile or sensory stimuli, and to systems utilizing combinations of stimulation types. The use of feedback control in the brainwave stimulation system overcomes the problem of a subject's brain adapting and becoming desensitized to brainwave stimulation, thereby enhancing the long-term effectiveness of the system. By adjusting as needed the frequency, intensity, and pattern of stimulation in response to detection of the subject's responses to stimulation, and/or by altering natural stimuli rather than merely providing modulated artificial stimuli, desensitization is avoided. Other suitable process control schema may alternatively be used, including feed-forward control, combined feedback/feed-forward control, model predictive control, and others. It further will be understood that in systems according to the disclosure using combinations of altered visual, aural, and/or tactile/sensory stimuli, the combined systems may modulated the stimuli at or with the same or different frequencies, patterns, and methods.

In addition to varying, modifying, and/or augmenting natural stimuli, and using feedback loops to do so, the system of the disclosure may further use pre-obtained information and data to optimize the brainwave stimulation. For example, the subject may receive brain scans at the outset of treatment or periodically or continuously during the course of treatment to assess the subject's individual needs and to tailor patterns of stimulation. Any or combinations of Magnetic Resonance Imaging (“MRI”), EEG, MEG, functional Magnetic Resonance Imaging (“MU”), and functional near-infrared spectroscopy (“fNIR”), and other tools may be used to determine the degree, type, effectiveness, and frequency of brainwave stimulation needed for a particular subject.

Subject input and feedback may further be used to modify and optimize the brainwave stimulation. For example, a subject may provide before or during brainwave stimulation feedback responses and/or preferences via an input device such as a PC computer, tablet, phone, mobile application, buttons, voice commands, web page or variations thereof.

A clinician may determine the degree, type, and frequency of brainwave stimulation for a particular subject based on the subject's performance as measured by questionnaires, tests, games, and/or other forms of assessment, either administered by a clinician or self-administered, either before, during, or after a course of brainwave stimulation treatment.

Embodiments of the system of the disclosure may also be configured to automatically adapt to various environmental factors using, e.g., a sensor, such as the microphone, camera, or membrane of the above-mentioned embodiments. If the sensor detects certain environmental conditions affecting, for instance, the type or degree of natural stimuli that are likely to be encountered, the control module can adapt the system accordingly. For example, if a camera or light sensor used in conjunction with a natural visual stimulation system detect that the light intensity has fallen, the system may adjust the patterns of the modulation of natural visual stimuli accordingly. If, in another embodiment, a microphone used in conjunction with a natural aural stimulation system detects that the subject is sleeping, the modulation of natural aural stimuli can be adjusted as appropriate.

The system may record the accumulated amount and/or the intensity of the stimuli that the subject has been exposed to over a period of time to adjust and report the dose of stimuli. For example, a particular subject may reach the desired daily dose of stimuli during bright daylight quicker than if staying in low-light conditions. The measure of the accumulated environmental stimuli can be used to increase, reduce or stop the stimulation for the day and to report the dose stimuli that the subject received over the period.

In other embodiments of the system for brainwave stimulation using altered natural stimuli of the disclosure, a central control unit is used by the system to control a set of natural stimulation units simultaneously, allowing the stimuli to change over time. The simultaneous control of multiple systems allows the stimulation delivered to the subject to be dynamic, changing the intensity, type, and frequency of stimulation delivered to different natural stimuli inputs at different times. Thus, for example, the system may emphasize stimulation delivered aurally during the day and focus on visual stimulation during quieter evening hours. The dynamic simultaneous control of multiple stimulation systems is advantageously adaptable to the subject's individual needs and also helps to prevent the subject's brain from becoming desensitized to a single type of stimulation. The system is further adaptable to accommodate a particular subject's lifestyle with minimized disruption such that a subject's preferred or customary activities are accounted for as the system is used.

For example, a particular subject normally may be exposed to visual stimuli that may be modulated using the system during work hours but may be exposed to aural stimuli that may be successfully modulated in the evening hours. Other subjects may participate in activities that correspond to tactile or sensory stimulation during the day but may respond well to visual stimulation at night. The system according to embodiments of the disclosure may be adapted for variations between subjects and over time.

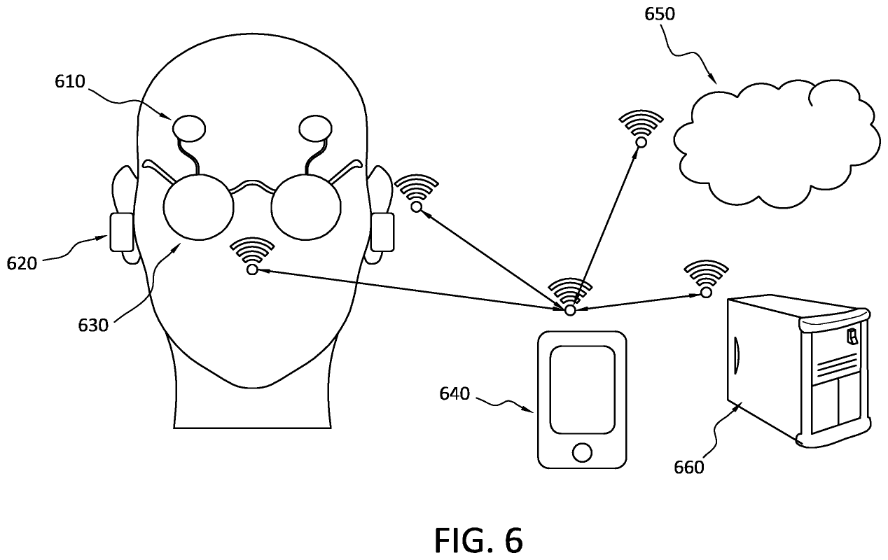

In any of the foregoing embodiments, the control module in the stimulating device may be interlinked with an external control unit over a wired or wireless communication link as shown in the diagram of FIG. 6. A system 600 according to another embodiment of the disclosure includes a subject wearing electrodes 610 on their forehead which provide EEG signals. A stimulation unit 620 for aural stimulation and a stimulation unit 630 for visual stimulation may contain wireless links and may communicate with each other, with a local control unit, and/or an external control unit. A mobile device 640 may communicate with the stimulation units 620 and 630 and may further communicate with external processors and other services on a cloud 650 or locally 660. The system 600 may include more or fewer stimulation units than shown in FIG. 6 and it is to be understood that one stimulation unit may contain or alter more than one sensory stimulus, such as both visual and aural stimulation in the same system.

The network shown in FIG. 6 allows the control and processing of the stimulation to take place anywhere in the network and thereby reduces the complexity of the functions that need to be performed locally on the wearable stimulation units 620, 630, reducing the cost and complexity of components and increasing the processing resources available to a system according to the disclosure. The stimulation units 620, 630 may therefore in one embodiment contain all the necessary functions for signal measuring, recording, processing and controlling logic within the stimulating unit itself. In another embodiment the stimulation units 620, 630 may fully rely on external control units for any of those functions.

In other embodiments the stimulation units 620, 630 may be a part of a system where the stimulating devices 620, 630 partly rely on external control units for any suitable functions. For example, the system 600 may be arranged such that the local control unit performs a predetermined part of the control computations and such that the external control unit performs a different predetermined part of the control computations.

In the embodiment shown in FIG. 6, the external control unit is a mobile device 660 using a wireless link with the stimulating devices 620 and 630 to control and/or receive signals from the stimulating unit. This mobile device may be further linked over network to an external servers 650 and 660 and may receive control information from the external servers 650, 660 on the operation functions of the device and may upload signals and status information from the stimulating device to the server. In another embodiment, the stimulation units 620 and 630 may have wireless modules for communicating directly with a local server 660 or cloud service 650 where the mobile device 640 is no longer needed as a bridge between the stimulation units and the servers.

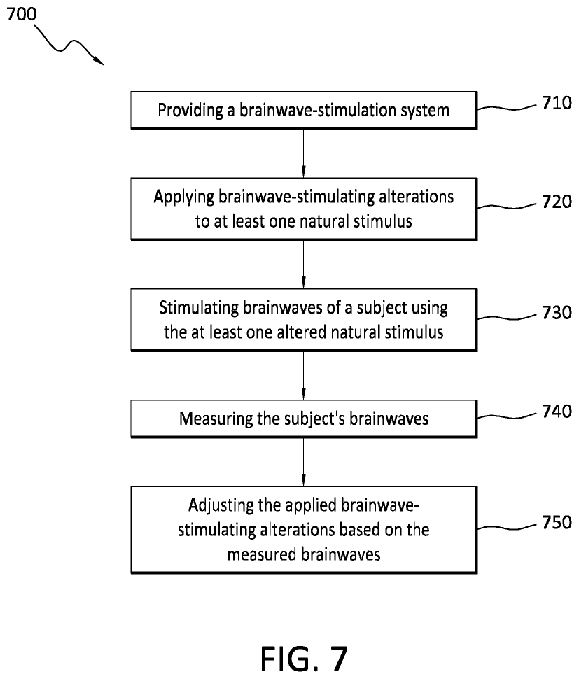

A method according to the disclosure may include the steps shown in method 700 in FIG. 7. The method 700 includes a first step 710 of providing a brainwave-stimulation system according to the disclosure. As discussed above, the brainwave-stimulation system may be arranged for altering natural stimuli such as visual, aural, tactile or sensory, or other stimuli and may be arranged for allowing a subject using the system to receive brainwave stimulation while engaging in normal, daily activities.

The method 700 includes a second step 720 of applying brainwave-stimulating alterations to at least one natural stimulus. In embodiments where the system provided in step 710 is directed to visual stimuli, the system may at step 720 modulate visual stimuli with frequencies for brainwave stimulation, such as in the range of 20-80 Hz. In embodiments where the system provided in step 710 is directed to aural stimuli, the system may similarly at step 720 modulate intercepted aural stimuli with frequencies for brainwave stimulation.

The method 700 further includes a third step 730 of stimulating a subject's brainwaves using the at least one altered natural stimulus. The third step 730 may be performed as described in embodiments of the disclosure, including by utilizing active-shutter lenses for visual stimulation, by feeding altered natural soundwaves to a subject's ear, by modulating touch pressure against a part of the subject's body, or by any other suitable procedure.

The method 700 optionally includes a fourth step 740 of measuring the subject's brainwaves. As described in the foregoing embodiments, the subject's brainwaves may be measured by, for example, EEG, MEG, or other suitable tools. The subject's measured brainwaves may advantageously verify the effectiveness of the stimulation treatment.

The method 700 optionally includes a fifth step 750 of adjusting the applied brainwave-stimulating alternations based on the measured brainwaves. The fifth step 750 may be performed as a part of a process control scheme, such as feedback or feed-forward control. The fifth step 750 may advantageously ensure that the subject's brain does not become desensitized to the stimulation treatment by varying the stimulation when the measured brainwaves indicate a diminished response to the stimulation treatment, particularly over time.

### EXPERIMENTAL RESULTS

An experiment was conducted to assess the brainwave-stimulation effects of embodiments of a system and method for brainwave stimulation using altered natural stimuli as described herein. A system according to embodiments of the present disclosure was prepared including visual and aural stimulation, EEG sensors for measuring stimulation efficiency of the system, and a control module connected to the stimulation units.

The visual-stimulation system was prepared with active-shutter glasses as described above in regards to the embodiment of FIG. 1. The control module utilized an Arduino Beetle attached to the glasses frame and configured to cause the lenses of the active-shutter glasses to blink at 40 Hz and with a 50% open/close ratio.

The aural-stimulation system was prepared according to the embodiment of FIG. 2. The EEG sensors were prepared with a high-density eego mylab™ EEG recorder having 33 channels and commercially available from ANT Neuro of Hengelo, Netherlands. The data recorded by the eego mylab™ were processed using Matlab.

The experiment included periods without stimulation at the beginning and end of recording sessions to measure any crosstalk or interference between the stimulating systems and the recorded EEG. The experiment was conducted on five healthy Caucasian males aged 24-82 years.

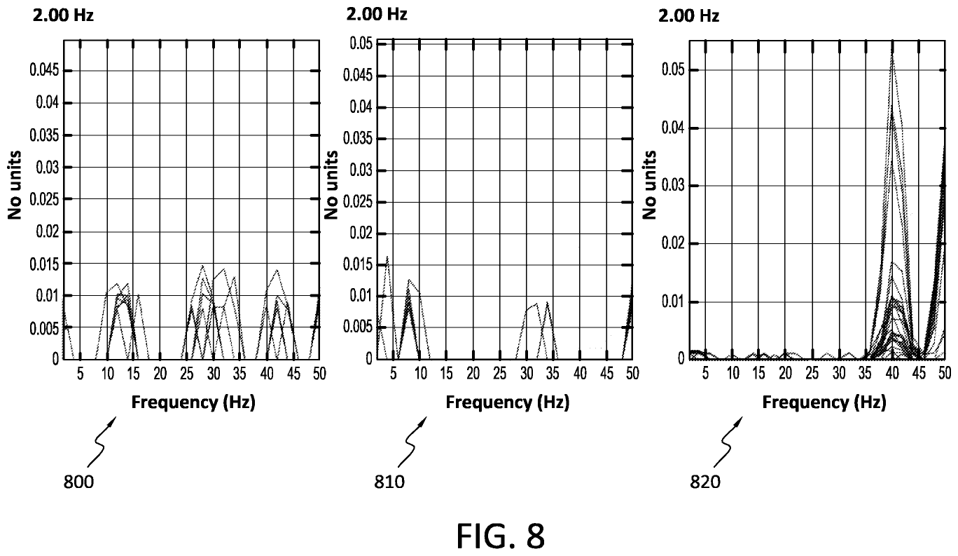

Alignment, including phase alignment or coherence, between the applied stimulation patterns and the subjects' EEG data was calculated in Matlab for different natural stimulus content and time periods and are shown in FIGS. 8-12C as a function of the applied frequency (shown on the x axis). Coherence is a unitless measure of the alignment between two signals. The coherence may range from 0 (indicating zero alignment between the two signals) to 1 (indicating perfect or 100% alignment between the two signals). A coherence value in the range of 0.5 may indicate that the signals come partially from the same source but are mixed with signals that come partially from different sources.

As shown in FIG. 8, first coherence obtained from the EEG sensors was compared between periods of silence 800 (corresponding to the aural-stimulation system being deactivated), white noise 810 (in which the aural-stimulation system is active but only presents unmodulated white noise), and audio content 820 modulated at a frequency of 40 Hz (including modulated aural content such as music, audiobook readings and nature sounds). As seen in FIG. 8, there is no indication of any direct crosstalk from the electronics between silence 800 and white noise 810, indicating no contamination in the data from the stimulation equipment. The increased coherence between the brainwave-stimulation signal applied by the aural-stimulation system and the recorded EEG signals from the subjects observed in 820 is attributed therefore to brainwave stimulation and is concentrated at the modulation frequency of 40 Hz.

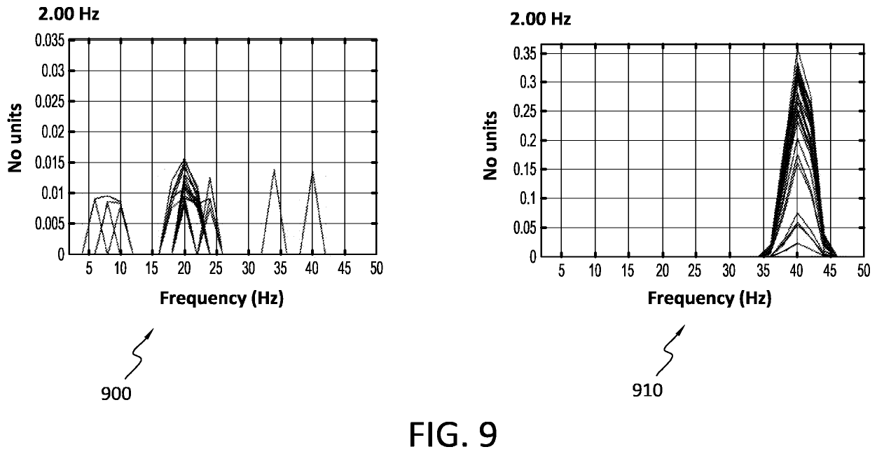

The results in FIG. 9 similarly demonstrate the effect of visual stimulation. Coherence measurements corresponding to the subjects' eyes being blocked are shown at 900. Coherence measurements corresponding to visual stimulation modulated at 40 Hz is shown at 910. As in FIG. 8, the effects of the visual stimulation is observed clearly at 40 Hz without indications of crosstalk from the visual-stimulation equipment between 900 and 910.

In addition to measuring the coherence between the stimulation signals and the subjects' brainwaves using the aural and visual stimulation systems as shown above in FIGS. 8 and 9, the stimulation was tested using both aural- and visual-stimulation systems continuously for 85 minutes, with measurements and calculations of the coherence performed for the first 30 minutes, the last 30 minutes, and over the entire 85 minutes.

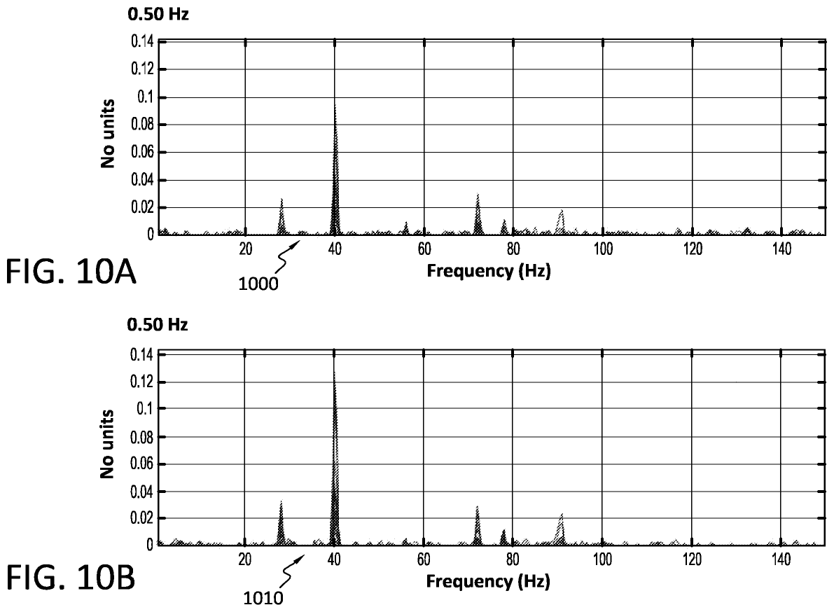

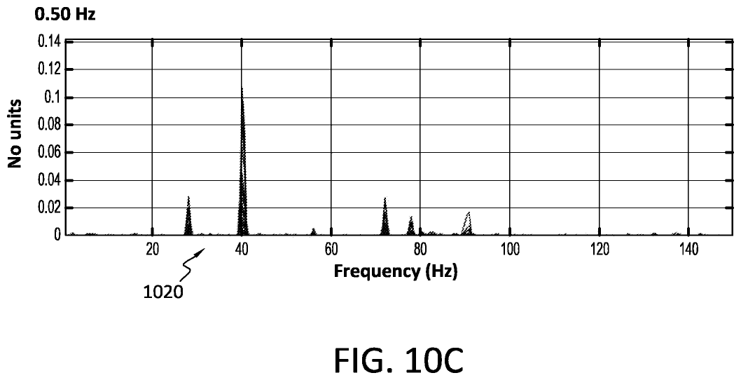

The results of the continuous testing for the aural stimulation system using modulated music are shown in FIGS. 10A-10C. FIG. 10A shows the coherence for the first 30 minutes of 40 Hz modulated music content, FIG. 10B shows the coherence for the last 30 minutes, and FIG. 10C shows the coherence for the entire 85 minutes. As seen from the consistent coherence observed between FIGS. 10A, 10B, and 10C, the coherence at 40 Hz is significant through all periods that were measured. Because little to no reduction in coherence is measured toward the end of the testing period, it is concluded that there was no adaptation by the subjects' brain to the modulated music-based aural stimulation.

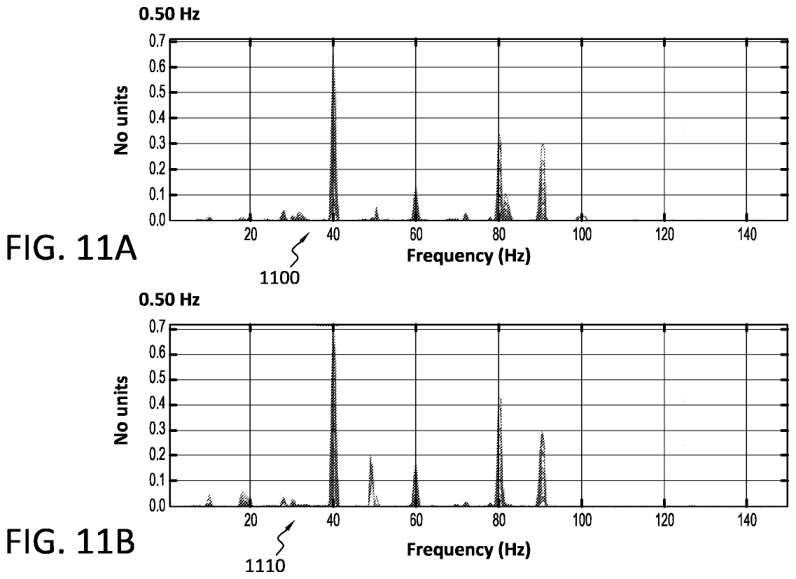

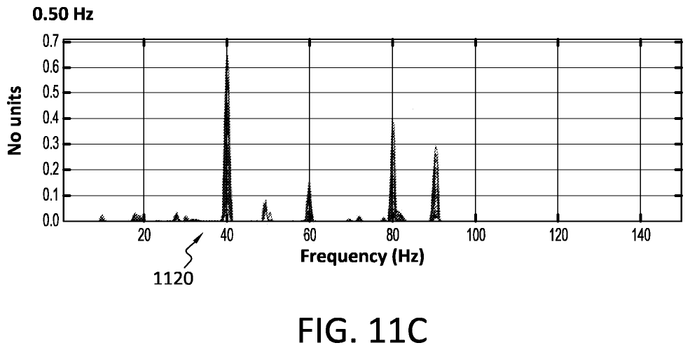

The results of the continuous testing for the aural stimulation using modulated white noise rather than music are shown in FIGS. 11A-11C. FIG. 11A shows the coherence for the first 30 minutes of 40 Hz modulated white noise, FIG. 11B shows the coherence for the last 30 minutes of 40 Hz modulated white noise, and FIG. 11C shows the coherence for the entire 85 minutes. Similar to the observations in FIGS. 10A-10C above, and as seen from the consistent coherence observed between FIGS. 11A, 11B, and 11C, the coherence at 40 Hz is significant through all periods that were measured. Because little to no reduction in coherence is measured toward the end of the testing period, it is concluded that there was no adaptation by the subjects' brain to the modulated white-noise aural stimulation.

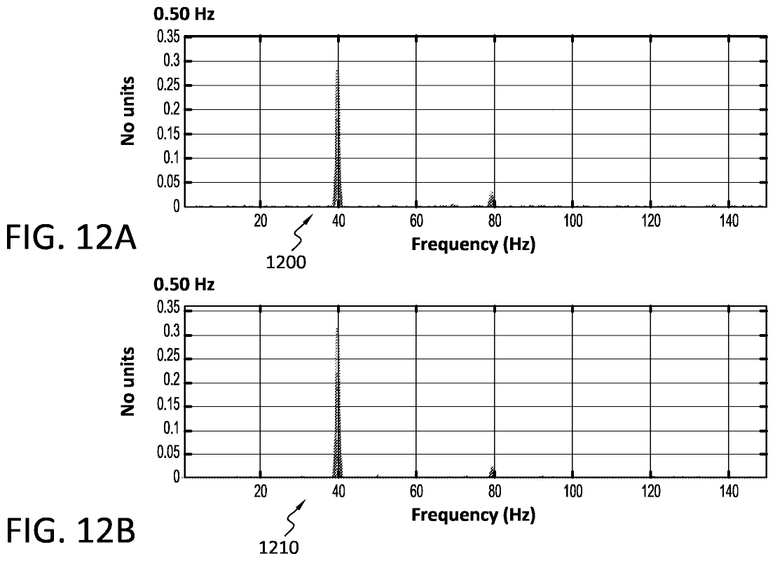

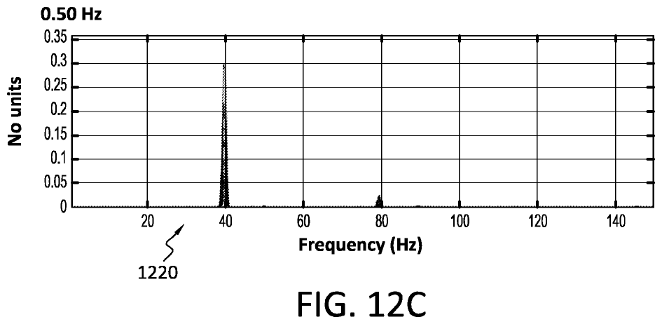

The results of the continuous testing for visual stimulation using modulated dash-cam driving video are shown in FIGS. 12A-12C. FIG. 12A shows the coherence for the first 30 minutes of 40 Hz modulated dash-cam driving video, FIG. 12B shows the coherence for the last 30 minutes of 40 Hz modulated dash-cam driving video, and FIG. 12C shows the coherence for the entire 85 minutes. As with FIGS. 10A-10C and 11A-11C above, the consistent coherence observed across each of FIGS. 12A-12C indicates that the coherence at 40 Hz is consistent and significant through all periods that were measured. It is concluded that there was no adaptation by the subjects' brain to the modulated dash-cam driving video stimulation.

These and other embodiments of the present disclosure overcome the deficiencies of existing brainwave stimulation systems by allowing a subject to receive brainwave stimulation during everyday activities (such that the stimulation is sufficiently long to be effective for treatment of neurological pathologies or for receiving other benefits from brainwave stimulation) and without brain adaptation/desensitization to the brainwave stimulation. The embodiments of the system accomplish this by providing systems for visual, aural, and/or tactile brainwave stimulation that intercept and alter natural stimuli. The subject is thus free to engage in normal daily activities while simultaneously receiving brainwave stimulation.

Although this disclosure describes certain exemplary embodiments and examples of a system and method for brainwave stimulation using altered natural stimuli, it nevertheless will be understood by those skilled in the art that the present disclosure extends beyond the specifically disclosed brainwave stimulation system embodiments to other alternative embodiments and/or users of the disclosure and obvious modifications and equivalents thereof. It is intended that the scope of the present disclosure should not be limited by the particular disclosed embodiments described above, and may be extended to other forms of neurological treatment, and other applications that may employ the features described herein.

It is understood that alternatives and modifications of these embodiments, such as those suggested by others, may be made to fall within the scope of the disclosure.

The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

1.-18. (canceled)

19. A system for brainwave stimulation of a subject using altered natural stimuli, the system comprising:
a natural stimulus modulator arranged for intercepting, measuring and modifying at least one natural stimulus signal with at least one brainwave-stimulation frequency for brainwave stimulation and for delivering the modulated at least one natural stimulus signal;
a control module arranged for creating a control signal for controlling the natural stimulus modulator;
wherein the at least one natural stimulus signal is obtained and modulated with the at least one brainwave-stimulation frequency during normal activities;

20. The system for brainwave stimulation according to claim 19, the system further comprising at least one brainwave sensor arranged for obtaining and transmitting brainwaves to the control module; and
characterized further in that the control module is arranged for maintaining a substantially constant level of brainwave stimulation by adjusting the at least one brainwave-stimulation frequency.

21. The system for brainwave stimulation according to claim 19, wherein the natural stimulus modulator modulates the at least one natural stimulus signal for at least evoking brainwaves at frequencies between 20 Hz and 80 Hz.

22. The system or brainwave stimulation according to claim 19, wherein the system simultaneously modulates and delivers two or more natural stimulus signals corresponding to different senses, including vision, auditory, and tactile or sensory.

23. The system for brainwave stimulation using altered natural stimuli according to claim 19, wherein the control module calculates a relationship between the modulated at least one natural stimulus signal and the brainwaves and adjusts the control signal according to the relationship.

24. The system for brainwave stimulation using altered natural stimuli according to claim 23, wherein the control module utilizes artificially learned methods to alter the control signal.

25. The system for brainwave stimulation using altered natural stimuli according to claim 20, wherein the at least one brainwave sensor comprises electrodes arranged to measure the brainwaves via EEG.

26. The system for brainwave stimulation using altered natural stimuli according to claim 20, wherein the at least one brainwave sensor comprises magnetic sensors arranged to measure the brainwaves via MEG.

27. The system for brainwave stimulation using altered natural stimuli according to claim 19, wherein the natural stimulus modulator further comprising a controllable transparent element arranged for modulating the at least one natural stimulus signal.

28. The system for brainwave stimulation using altered natural stimuli according to claim 19, the system further comprising a sensor configured to measure the at least one natural stimulus signal and deliver the measurement of the at least one natural stimulus signal to the control module.

29. The system for brainwave stimulation using altered natural stimuli according to claim 19, wherein the natural stimulus modulator is further arranged to adjust a quantity of the at least one natural stimulus signal that is delivered.

30. The system for brainwave stimulation using altered natural stimuli according to claim 28, wherein the control module adjusts a frequency, intensity, or form of the control signal according to environmental parameters measured by the sensor.

31. The system for brainwave stimulation using altered natural stimuli according to claim 19, wherein the control module further is arranged for providing a synthesized signal in addition to the altered at least one natural stimulus signal.

32. A system for brainwave stimulation of a subject using altered natural stimuli, the system comprising:
a natural stimulus modulator in the form of glasses using variable transparent material capable of modulating at least a natural visual stimulus signal with at least one brainwave-stimulation frequency;
a control module configured to create a control signal for controlling the natural stimulus modulator; and
wherein the natural stimulus modulator is arranged for modifying the at least one natural stimulus signals at least evoking brainwaves at frequencies between 20 and 80 Hz;

33. A system for brainwave stimulation of a subject using altered natural stimuli, the system comprising:
a natural stimulus modulator using microphone and speaker capable of modulating at least a natural aural stimulus signal with at least one brainwave-stimulation frequency;
a control module configured to create a control signal for controlling the natural stimulus modulator; and
wherein the natural stimulus modulator is arranged for modifying the at least one natural stimulus signals at least evoking brainwaves at frequencies between 20 and 80 Hz;

34. The system for brainwave stimulation using altered natural stimuli according to claim 19, wherein the stimulating control functions are determined partially in the control module and partially received over a wired or wireless link from an external control module.

35. The system for brainwave stimulation using altered natural stimuli according to claim 19, wherein the at least one natural stimulus modulator communicates wirelessly to an external cloud server over a direct or indirect communication link.

36. A method for brainwave stimulation of a subject using altered natural stimuli, the method comprising the steps of:
providing a brainwave stimulation system according to claim 19;
modulating at least two natural stimulus signals comprising respectively natural visual and aural stimulus signals with at least a brainwave-stimulating frequency;
stimulating brainwaves by delivering the at least two altered natural stimulus signals;
measuring the brainwaves;
determining a relationship between the measured brainwaves and the modulated at least two natural stimulus signals; and
adjusting the applied brainwave-stimulating frequency based on the determined relationship to maintain a substantially constant level of brainwave stimulation;
wherein the at least two natural stimulus signals are obtained and modulated with the at least one brainwave-stimulating modulation during normal activities.
